(19) United States
(12) Patent Application Publication
(10) Pub. No.: US 2007/0134730 A1
(43) Pub. Date: Jun. 14, 2007

(54) Screening method, screening program and screening device

(75) Inventors: Toshihide Ezoe, Kanagawa (JP); Takayuki Yamada, Kanagawa (JP); Hirohiko Tsuzuki, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Minato-ku (JP)

(21) Appl. No.: 11/637,837

(22) Filed: Dec. 13, 2006

(30) Foreign Application Priority Data
Dec. 13, 2005 (JP) 2005-359619

(51) Int. Cl. G01N 33/53; G06F 19/00

(52) U.S. Cl. 435/7.1; 702/19

(57) ABSTRACT

The invention provides a screening method for selecting a particular candidate molecule by using binding amount data between a target molecule and the candidate molecule, the data being obtained by supplying the candidate molecule of study to a measurement region composed of an immobilization membrane on which the target molecule is immobilized, the screening method comprising: obtaining the binding amount data during activity by supplying the candidate molecule to a measurement region in which the target molecule not subjected to activity lowering treatment that lowers physiological activity is immobilized, obtaining the binding amount data during low activity by supplying the candidate molecule to a measurement region in which the target molecule that is subjected to activity lowering treatment that lowers physiological activity is immobilized, and extracting a candidate molecule having a specified difference between the binding amount data during the activity and the binding amount data during the low activity.

### CROSS REFERENCES TO RELATED APPLICATIONS

This invention claims priority under 35 USC 119 from Japanese Patent Application No. 2005-359619 the disclosure of which is incorporated by reference herein.

### BACKGROUND OF THE INVENTION

1. Field of the invention

The present invention relates to a screening method, a screening program and a screening device. More specifically, the invention relates to a screening method, a screening program and a screening device, which extract a specified candidate molecule by use of binding between a target molecule and a candidate molecule.

2. Description of the Related Art

A variety of biosensors have been proposed as devices for measuring the interaction between physiologically active substances such as proteins and specified compounds. Of these, the surface plasmon sensor is known as a measuring device utilizing an evanescent wave (see Japanese Patent No. 3294605). In general, the surface plasmon sensor includes a prism, a metal membrane on which a physiologically active substance placed on one face of the prism is immobilized, a light source that generates light beams, an optical system that causes light beams to put thereto at various angles with respect to the prism so as to obtain the total reflection conditions at the interface between the prism and the metal membrane, and light-detecting means for detecting the intensity of the light beams totally reflected at the interface, and carries out measurements on the physiologically active substance on the basis of the detection results of the light-detecting means. In measurement by means of this surface plasmon sensor, a compound solution is provided with a physiologically active substance immobilized on the metal membrane and light beams are put to the face opposite to the side with which the compound solution on the metal membrane to measure the interaction between the physiologically active substance and the compound in the compound solution, based on the information of the refractive index obtained from the reflected light.

Incidentally, the surface plasmon sensor can precisely detect fine conditions between molecules on a metal membrane, so the sensor is suitable for massive-scale screening of selecting a large number of candidate molecules when a particular physiologically active substance is immobilized on a metal membrane as a target molecule to determine as an index whether or not a molecule can specifically bind to the target molecule.

Some candidate molecules, however, non-specifically bind to a target molecule. The surface plasmon sensor detects changes of binding conditions between compounds as described above, and thus changes in such binding conditions only cannot distinguish between specific binding and non-specific binding between candidate and target molecules. As such, when a large amount of screening is carried out, there is the problem that it takes time to narrow down the candidate molecules.

For the purpose of reduction of the number of non-specific bindings mentioned above, it has been performed to add a high concentration of a surfactant such as Tween 20 or polyoxyalkylenearyl ether to a buffer or the like to be used in the measurement (e.g., Japanese Patent Laid-Open Nos. 58-187862, 61-260163 and 6-300759).

However, the method of using a buffer that contains a high concentration of a surfactant causes a high concentration of the surfactant to be always present in the measurement system, sometimes leading to loss of the measurement stability due to its adsorption to or desorption from the sensor surface. In addition, a large amount of buffer or the like containing a surfactant needs to be prepared.

### SUMMARY OF THE INVENTION

The invention has been made in view of the above circumstances and provides a screening method, a screening program and a screening device.

A first aspect of the invention provides a screening method for selecting a particular candidate molecule by use of data of binding amount between a target molecule and the candidate molecule, the data being obtained by supplying the candidate molecule of study to a measurement region composed of an immobilization membrane on which the target molecule is immobilized, the screening method comprising: obtaining the data of binding amount during activity by supplying the candidate molecule to a measurement region in which the target molecule not subjected to activity lowering treatment that lowers physiological activity is immobilized, obtaining the data of binding amount during low activity by supplying the candidate molecule to a measurement region in which the target molecule that is subjected to activity lowering treatment that lowers physiological activity is immobilized, and extracting a candidate molecule having a specified difference between the data of binding amount during the activity and the data of binding amount during the low activity.

### BRIEF DESCRIPTION OF THE DRAWINGS

FIG. 1 is a perspective view of the whole of a biosensor concerning an embodiment of the invention.

FIG. 2 is a perspective view of a sensor stick concerning an embodiment of the invention.

FIG. 3 is an exploded perspective view of the sensor stick of the present embodiment.

FIG. 4 is a sectional view of the liquid flow channel part of 1 of the sensor stick of the present embodiment.

FIG. 5 is a drawing in which light beams are put to the measurement region and the reference region of the sensor stick of the present embodiment.

FIGS. 6A to 6C are side views of a pipet part constituting a liquid supplying part concerning an embodiment of the invention.

FIG. 7 is a schematic view of the vicinity of the optical measurement unit of the biosensor concerning an embodiment of the invention.

FIG. 8 is a schematic block diagram of the control unit and its vicinity concerning the embodiment of the invention.

FIG. 9 shows a flow chart of a screening process concerning a First Embodiment of the invention.

FIG. 10 shows an example of a candidate molecule list concerning the embodiment of the invention.

FIG. 11 shows a flow chart of a screening process concerning a Second Embodiment of the invention.

### DETAILED DESCRIPTION OF THE INVENTION

According to the invention, there are provided a screening method, a screening program and a screening device that are able to simply and easily and efficiently extract a candidate molecule capable of specifically binding to a target molecule, by efficiently removing a candidate molecule capable of non-specifically binding to a target molecule.

The screening method of the invention is a screening method for selecting a particular candidate molecule by use of data of binding amount between a target molecule and the candidate molecule, the data being obtained by supplying the candidate molecule of study to a measurement region composed of an immobilization membrane on which the target molecule is immobilized. The screening method includes obtaining the data of binding amount during activity by supplying the candidate molecule to a measurement region in which the target molecule not subjected to activity lowering treatment that lowers physiological activity is immobilized, obtaining the data of binding amount during low activity by supplying the candidate molecule to a measurement region in which the target molecule that is subjected to activity lowering treatment that lowers physiological activity is immobilized, and extracting a candidate molecule having a specified difference between the data of binding amount during the activity and the data of binding amount during the low activity.

The screening program of the invention is a screening program for selecting a particular candidate molecule by use of data of binding amount between a target molecule and a candidate molecule, the data being obtained by supplying the candidate molecule of study to a measurement region composed of an immobilization membrane on which the target molecule is immobilized. The screening program causes a computer to execute, the screening program comprising: calculating data of binding amount during activity, based on binding amount between the target molecule and the candidate molecule supplied to a measurement region in which the target molecule not subjected to activity lowering treatment that lowers physiological activity is immobilized, calculating data of binding amount during low activity, based on binding amount between the target molecule and the candidate molecule supplied to a measurement region in which the target molecule that is subjected to activity lowering treatment that lowers physiological activity is immobilized, and extracting a candidate molecule showing a specified difference between the data of binding amount during activity and the data of binding amount during low activity.

Furthermore, the screening device of the invention is a screening device for selecting a particular candidate molecule by use of data of binding amount between a target molecule and a candidate molecule, the data being obtained by supplying the candidate molecule of study to a measurement region composed of an immobilization membrane on which the target molecule is immobilized. The screening device comprises a measuring unit that obtains the data of binding amount by supplying a candidate molecule to the measurement region, an activity lowering treatment mechanism that lowers the physiological activity of the target molecule by supplying an activity lowering treatment solution to the measurement region, a memory that stores for every candidate molecule the data that is obtained from the measuring unit of the active binding amount of the candidate molecule to the target molecule not subjected to the activity lowering treatment, and stores for every candidate molecule data that is obtained from the measuring unit at low activity of the binding amount of an candidate molecule to a low activity target molecule after the activity lowering treatment; and extracting unit that extracts a candidate molecule having a specified difference between the data of active binding amount stored in the memory and the low activity measurement data.

The invention carries out activity lowering treatment that lowers physiological activity on a target molecule to be a target, for the purpose of screening a candidate molecule of study. Even though a candidate molecule can specifically bind to a target molecule, the candidate molecule cannot maintain the amount of intrinsic binding to a target molecule after such activity lowering treatment. Therefore, the data of binding amount obtained for a target molecule after activity lowering treatment (data of binding amount during low activity) fundamentally indicates a value lower than the data of binding amount obtained for a target molecule without activity lowering treatment (data of binding amount during activity), resulting in a specified difference between these data. On the other hand, for a candidate molecule that non-specifically binds to a target molecule, these data do not indicate a specified difference since the target molecule binds independently of the height of activity of the target molecule. Accordingly, on the basis of such specified differences, a candidate molecule that specifically binds a target molecule can efficiently be screened, with a candidate molecule non-specifically binding to a target molecule definitely removed.

In this case, a threshold for selecting a candidate molecule can be set in advance. This makes it possible to further efficiently carry out screening.

The threshold may be a threshold to be compared with the data of binding amount during low activity, or a threshold to be compared with the data of binding amount during activity. When a threshold compared with the data of binding amount during low activity is used, a candidate molecule showing the data of binding amount during low activity of the threshold or less, or a candidate molecule showing the data of binding amount during low activity of less than the threshold amount is extracted. On the other hand, when a threshold compared with the data of binding amount during activity is used, a candidate molecule showing the data of binding amount during activity of the threshold amount or more, or a candidate molecule showing the data of binding amount during activity exceeding the threshold amount is extracted. Moreover, a threshold compared with the difference between the data of binding amount during activity and the data of binding amount during low activity may be acceptable. In this case, a candidate molecule in the case where the difference between the data of binding amount during activity and the data of binding amount during low activity is the threshold amount or more, or the amount exceeding the threshold is extracted.

In an embodiment, a combination of plural thresholds may be used. This enables more precise screening of target candidate molecules.

Additionally, the extraction step may have a plurality of independent extraction steps that extract candidate molecules respectively using a plurality of thresholds that have been set. In this case, on a candidate molecule extracted by the first extraction step based on the threshold of 1, the second extraction based on the other threshold can be carried out. This makes it possible to reduce the number of candidate molecules to be subjected to the second extraction, enabling screening in a shorter time.

Furthermore, activity lowering treatment may be carried out on a target molecule subsequent to the obtaining of data of binding amount during activity. In this case, screening is performed on the basis on the data of binding amount during activity and the data of binding amount during low activity, both obtained for the same target molecule immobilized on an immobilization membrane, so a candidate molecule that specifically binds to a target molecule can be extracted more precisely.

Moreover, although activity lowering treatment can also be carried out on a target molecule prior to immobilization treatment depending on the kind of the target molecule, the treatment is preferably carried out on a target molecule immobilized on an immobilization membrane. This enables the obtaining of the data of binding amount during low activity from a target molecule, subsequent to activity lowering treatment, definitely immobilized on an immobilization membrane, even though the target molecule exhibits properties that hardly tend to be immobilized on an immobilization membrane as a result of activity lowering treatment.

In an embodiment, the immobilization membrane may be formed on one side of a metal membrane and is not formed on the other side, and obtaining the data of binding amount during activity and obtaining the data of binding amount during low activity may be carried out by a process including using attenuated total reflection generated by a light beams to the opposite side of the metal membrane on which the immobilization membrane is formed.

Such measurement may be conducted with means of the surface plasmon sensor and the leaky mode sensor.

In addition, obtaining the data of binding amount during activity and obtaining the data of binding amount during low activity may be carried out by a process including supplying the candidate molecule to the metal membrane on which the immobilization membrane is formed on which the target molecule is not immobilized is formed, the data being based on reference data obtained from the amount of attenuated total reflection generated by irradiating the light beams to the opposite side of the metal membrane to that on which the immobilization membrane on which the target molecule is not immobilized is formed.

The obtaining of reference data in this way makes it possible to correctly gain data of binding amount during activity and data of binding amount during low activity, leading to appropriate screening.

The target molecules and the candidate molecules in the invention may include physiologically active substances and organic or inorganic molecules capable of binding to these physiologically active substances including any one or more species of substances described as “Tennen Kohbunshi (Natural Macromolecules)” (Symbols 11001 to 11025) in Japanese Industrial Standards (JIS) K3611, saccharides, amino acids, nucleotides, lipids, hemes, quinones, proteins, RNA, DNA, phospholipids, and polysaccharides described in “I. Seitai Busshitu Gairon (Introduction To Biomolecules)” in Encyclopedic Databook of Biology (Asakura Publishing Co., Ltd.), proteins, amino acids, nucleic acids, lipids and enzymes described in “II. Seibutubusshitu To Taisha (Biomolecules And Metabolism)” in Bioscience Dictionary (Asakura Publishing Co., Ltd.).

In addition, the binding between a target molecule and a candidate molecule refers to a physical, chemical, or biochemical binding reaction.

### First Embodiment

The first Embodiment of the invention will be set forth in reference with Drawings hereinafter.

A biosensor 10 as a screening device of the embodiment is a surface plasmon sensor that measures the interaction between a physiologically active substance D as a target molecule and a candidate molecule, by making use of a surface plasmon generated on the surface of a metal membrane. The embodiment uses this biosensor 10 in order to screen a substance specifically binding to the physiologically active substance D from a large number of candidate molecules.

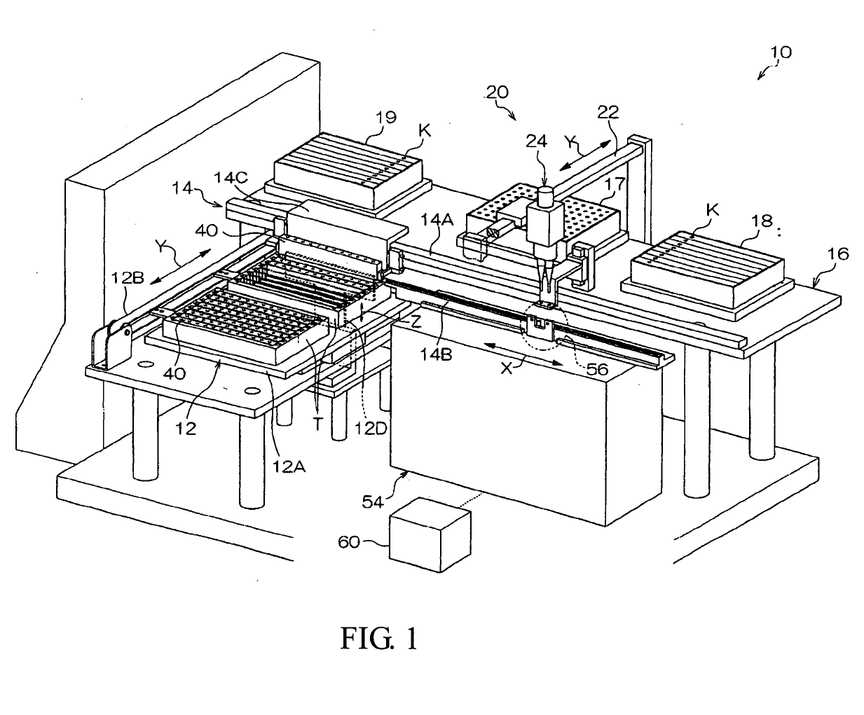

As shown in FIG. 1, the biosensor 10 includes a tray holding unit 12, a conveyance unit 14, a container mounting base 16, a liquid pumping unit 20, an optical measurement unit 54 and a control unit 60.

The tray holding unit 12 includes a mounting base 12A and a belt 12B. The mounting base 12A is fitted to the belt 12B put across the arrow Y direction, and is movable in the arrow Y direction by rotation of the belt 12B. On the mounting base 12A, two trays T are mounted in place. In the tray T, eight sensor sticks 40 are put. The sensor stick 40 is a chip on which the physiologically active substance D is immobilized, and will be described in detail later. Under the mounting base 12A, a pushing up mechanism (not shown) is disposed for pushing the sensor stick 40 up until the position of a stick holding member 14C to be described later.

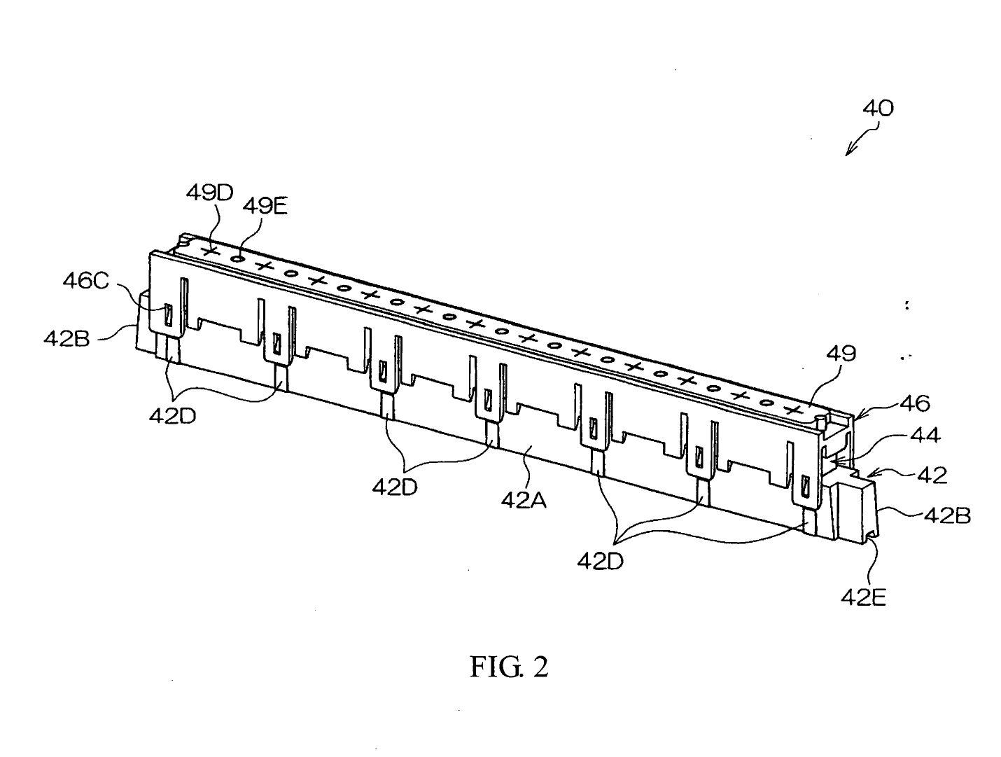

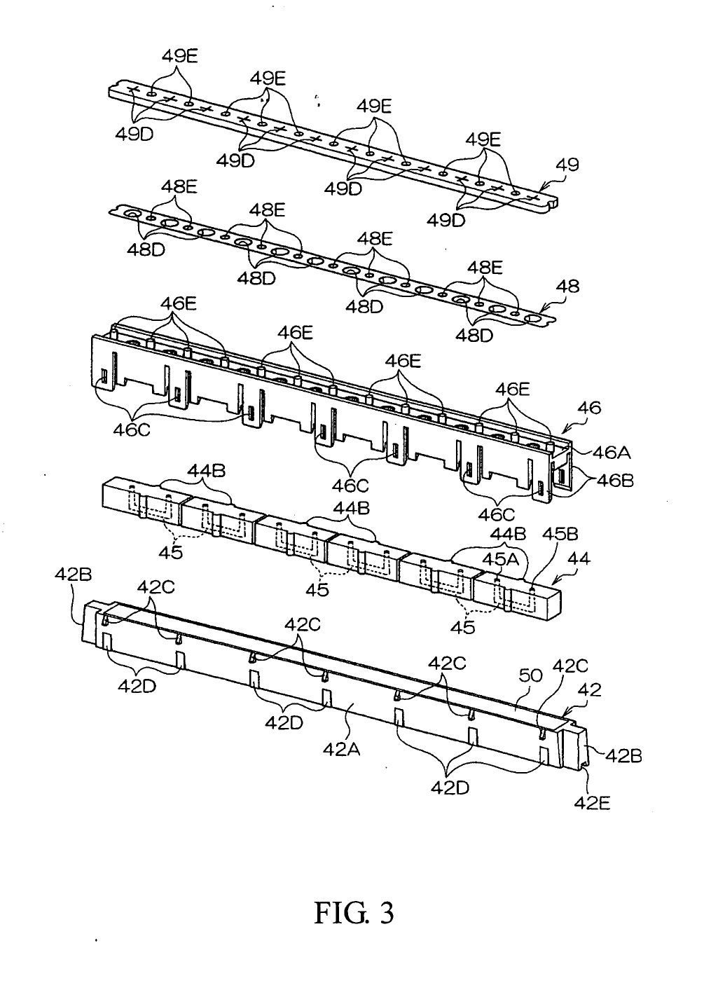

The sensor stick 40, as shown in FIG. 2 and FIG. 3, includes a dielectric block 42, a flow channel member 44, a holding member 46, an adhesion member 48 and an evaporation preventing member 49.

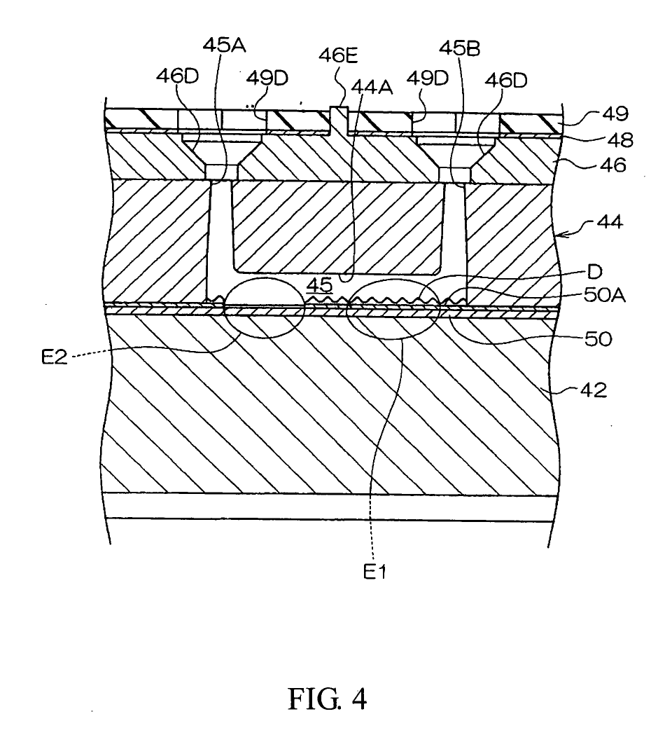

The dielectric block 42 is composed of a transparent resin or the like transparent to light beams, and includes a prism unit 42A having a stick shape with its cross section of a trapezoid and a held part 42B integrally formed relative to the prism unit 42A at both ends of the prism unit 42A. On the top face of a wider side of two faces parallel to each other of the prism unit 42A, a metal membrane 50 is formed as also shown in FIG. 4. The dielectric block 42 functions as a prism and, during the measurement by means of the biosensor 10, light beams are put into the prism unit 42A from one side of two sides opposite to and non-parallel to each other of the prism unit 42A and light beams totally reflected at the interface with the metal membrane 50 outgo from the other face.

On the surface of the metal membrane 50, as shown in FIG. 4, an immobilization membrane 50A is formed. The immobilization membrane 50A is provided to immobilize the physiologically active substance D on the metal membrane. The immobilization membrane 50A is selected depending on the kind of physiologically active substance D to be immobilized.

Examples of the immobilization membrane 50A that can be used include hydrogels such as agarose, dextran, carrageenan, alginic acid, starch, and cellulose, or derivatives thereof, e.g., carboxymethyl, or water swelled organic polymers, e.g., polyvinyl alcohol, polyacrylic acid, polyacrylamide, polyethylene glycol and the like. In particular, polyethylene glycol derivatives and dextran derivatives are preferably used from the viewpoint of physiological activity maintenance.

On the immobilization membrane 50A are formed a physiologically active substance D immobilized measurement region E1 and a reference region E2 in which the physiologically active substance D is not immobilized and for obtaining a reference signal during signal measurement in the measurement region E1. In other words, the reference region E2 is a region that is provided to correct the data obtained from the physiologically active substance D immobilized measurement region E1. This reference region E2 is formed when the above-described immobilization membrane 50A is formed. The forming method involves, for example, carrying out surface treatment on the immobilization membrane 50A and then deactivating the binding groups binding to the physiologically active substances D. This causes half of the immobilization membrane 50A to be the measurement region E1 and the remaining half to be the reference region E2.

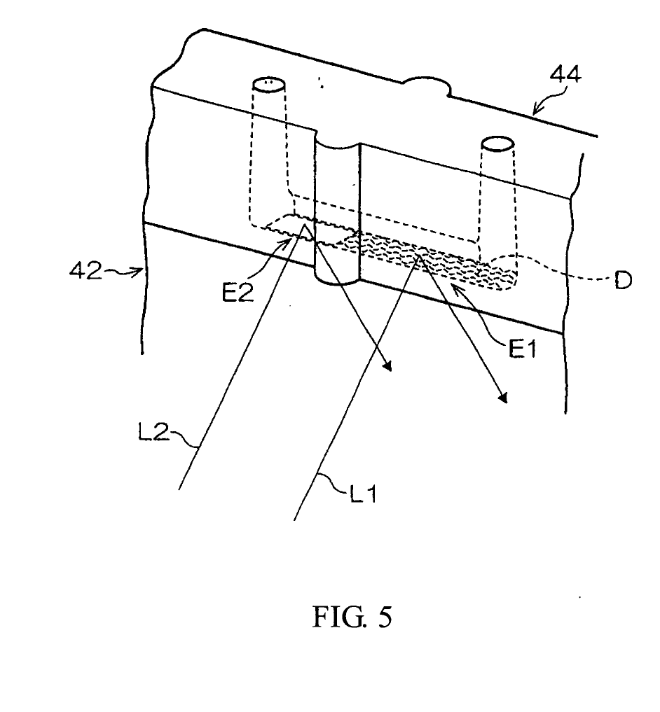

As also shown in FIG. 5, into the reference region E2 and the measurement region E1 located upstream from the reference region E2 are put light beams L2, L1, respectively.

On both sides of the prism unit 42A, an engaging convex part 42C engaging with the holding member 46, along the end edge of the upper side, and a vertical convex part 42D constructed on the extension of the virtual face perpendicular to the top face of the prism unit 42A, along the end edge of the lower side, each are formed in seven sites. Moreover, in the central part of the lower face, along the direction of the length, of the dielectric block 42 is formed an engaging groove 42E.

The flow channel member 44 has a rectangular parallelepiped shape slightly narrower in width than the dielectric block 42 and, as shown in FIG. 3, six of the flow channel members are placed in a line on the metal membrane 50 of the dielectric block 42. In the lower face of each flow channel member 44 is formed a flow channel groove 44A, and a liquid flow channel 45 is constructed, between the metal membrane 50, communicated with a supply unit 45A and a exhaust port 45B, formed on the top face of each flow channel member. Thus, one sensor stick has six independent liquid flow channels 45. In the side wall of the flow channel member 44, a convex 44B is formed, press fitted in a concave part (not shown) of the inside of the holding member 46, in order to ensure the adhesion with the holding member 46.

In addition, a liquid containing a protein is supposed to be supplied to the liquid flow channel 45, so material of the flow channel member 44 does not preferably have non-specific adsorptive property in a protein.

The holding member 46 is a long strip of material and includes a top face plate 46A and two side face plates 46B. In the side face plate 46B, an engaging hole 46C is formed that engages with an engaging convex part 42C of the dielectric block 42. The holding member 46 has six flow channel members 44 put between its two plates, is engaged with an engaging hole 46C and an engaging convex part 42C, and is fitted to the dielectric block 42. This causes the flow channel members 44 to be fitted to the dielectric block 42. In a position, of the top face plate 46A, opposite to a supply unit 45A and an exhaust port 45B is formed a taper-shape pipet insertion hole 46D that is narrower toward the flow channel member 44. Moreover, between two pipet insertion holes 46D that are placed side by side, a boss 46E for determining the position is formed.

To the top face of the holding member 46 is adhered the evaporation preventing member 49 through the adhesion member 48. In the position opposite to a pepit insertion hole 46D of the adhesion member 48 is formed a hole 48D for pipet insertion, and in the position opposite to a boss 46E is formed a hole 48E for determining the position is formed. In addition, in the position opposite to a pipet insertion hole 46D of the evaporation preventing member 49 is formed a slit 49D, a cut with a cross shape, and in the position opposite to a boss 46E is formed a hole 49E for determining the position. A boss 46E is inserted through holes 48E and 49E, and the evaporation preventing member 49 is adhered to the top surface of the holding member 52, so that a slit 49D of the evaporation preventing member is constructed so as to be opposite to a supplying port 44 and an exhaust port of the fluid channel member 49. When a pipet chip CP is not inserted, part of a slit 49D covers the corresponding supplying port 45B, preventing the evaporation of a liquid being supplied to the liquid flow channel 45.

As shown in FIG. 1, the conveyance unit 14 includes an upper part guide rail 14A, a lower part guide rail 14B and the stick holding member 14C. The upper part guide rail 14A and the lower part guide rail 14B are disposed in parallel with the arrow X direction perpendicular to the arrow Y direction, in the upper parts of the tray holding unit 12 and the optical measurement unit 54. To the upper part guide rail 14A is fitted the stick holding member 14C. The stick holding member 14C can hold the held part 42B of both ends of the sensor stick 40 and also is movable along the upper part guide rail 14A. The engaging groove 42E of the sensor stick 40 held in the stick holding member 14C is engaged with the lower part guide rail 14B, and the movement of the stick holding member 14C to the arrow X direction causes the sensor stick 40 to be conveyed to the measurement unit 56 on the optical measurement unit 54.

On the container mounting base 16 are mounted a compound solution plate 17, a buffer solution stock container 18, and a waste liquid container 19. The compound solution plate 17 is divided into 96 compartments and can stock a variety of compound solutions. The buffer solution stock container 18 includes containers 18A to 18E, and in the containers 18A to 18E an opening K that can insert the pipet chip CP to be described later is formed.

The buffer solutions that can be used include, for example, basic, well-known buffer solutions (Kagaku Binran Oyo Hen Revised 2nd Edition pp. 1312-1320) and buffers to which salts, metal ions (a magnesium ion, a potassium ion, a calcium ion, etc.), and a stabilizer (DTT, etc.). In particular, a PBS buffer (phosphoric acid buffer physiological saline), a Tris buffer, a HEPES buffer, and the like are preferably used.

The waste liquid containers 19 has a plurality of containers 19A to 19E, in which openings K to which pipet chips CP can be inserted are formed.

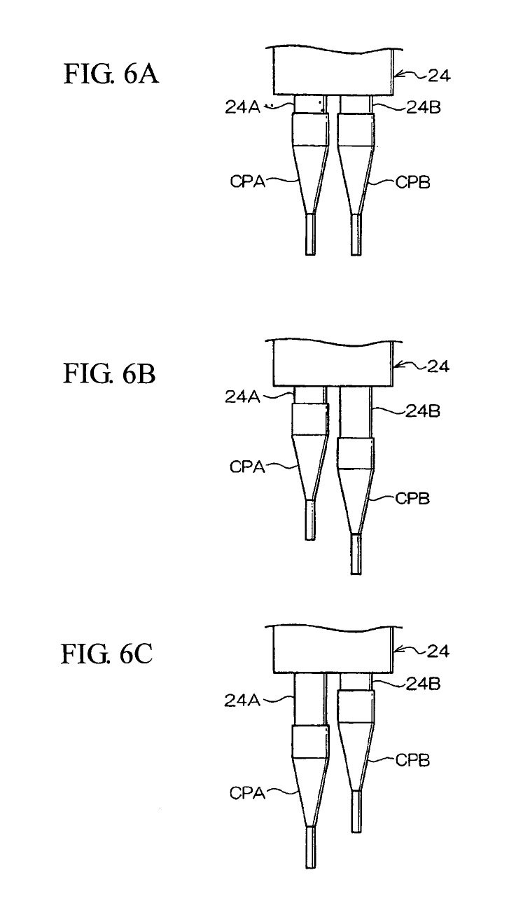

The liquid pumping unit 20 includes the upper part guide rail 14A, a crossing rail 22 put across in the arrow Y direction, which is upper part of the guide rail 16B, and a head 24. The crossing rail 22 is movable to the arrow X direction by means of a driving mechanism (not shown). In addition, the head 24 is fixed to the crossing rail 22, and movable to the arrow Y direction. Moreover, the head 24 is also movable to the vertical direction (arrow Z direction) by means of a driving mechanism (not shown). The head 24 includes, as shown FIG. 6, two pipet parts 24A, 24B. Pipet chips CP are fixed to the tips of the pipets 24A, 24B, and the lengths of their individual Z directions are adjustable (see FIG. 6A, FIG. 6B and FIG. 6C). Pipet chips CP are stocked in a pipet chip stocker (not shown), and, as required, are changeable.

In addition, in the embodiment, liquid supply to the sensor stick is carried out by means of a pipet chip CP, and instead of a pipet chip, an injection tube, one end of which is connected to each of the above solution plate, and the other end of which is connectable to the sensor stick 40, may be provided to supply a liquid by means of a liquid conveying pump.

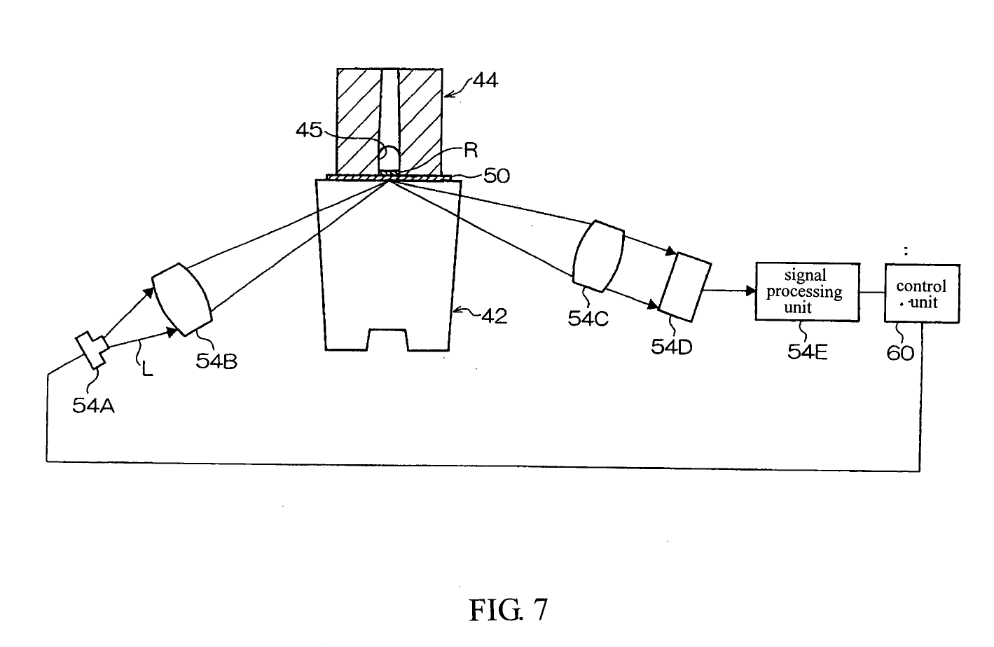

The optical measurement unit 54 includes, as shown in FIG. 7, a light source 54A, a first optical system 54B, a second optical system 54C, a light receiving unit 54D, and a signal processing unit 54E. From the light source 54A, light beams L in divergence are emitted. The light beams L become two light beams L1, L2 through the first optical system 54B, and are put into the measurement region E1 and the reference region E2 of the dielectric block 42 disposed in the measurement unit 56. In the measurement region E1 and the reference region E2, the light beams L1, L2 include a variety of incidence components relative to the interface relative to the metal membrane 50 and the dielectric block 42, and are put into the interface at an angle of more than the total reflection angle. The light beams L1, L2 are totally reflected at the interface between the dielectric block 42 and the metal membrane 50. These totally reflected beams L1, L2 are received in the light receiving part 54D through the second optical system 54C, and each of them is opto-electrically converted, and then light detection signals are put out to the signal processing part 54E. In the signal processing part 54E, specified treatment is carried out on the basis of the incident light detection signal to evaluate measurement data G1 of the measurement region E1 and reference data G2 of the reference region E2. These measurement data GI and the reference data G2 are put out to the control unit 60.

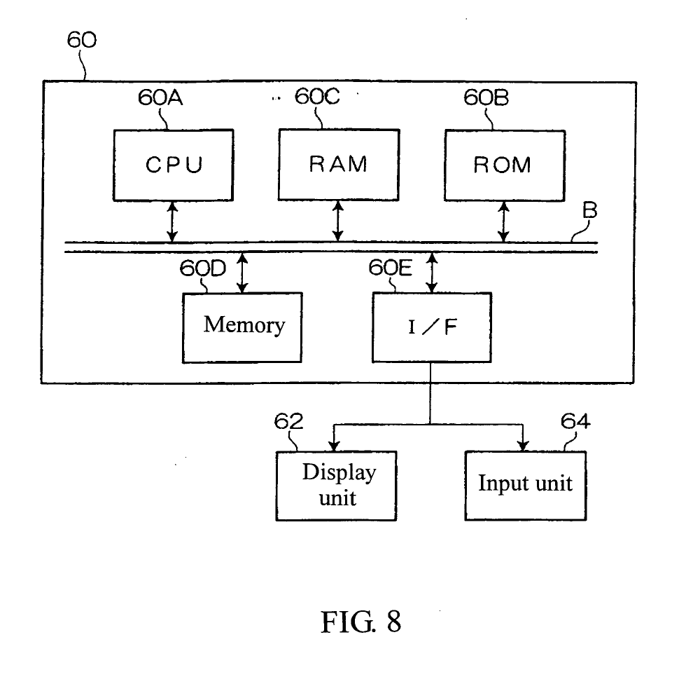

The control unit 60 has a function of controlling the whole of the biosensor 10 and, as shown in FIG. 7, is connected to the light source 54A, the signal processing unit 54E and a driving system (not shown) of the biosensor 10. The control unit 60 has, as shown in FIG. 8, CPU60A, ROM60B, RAM60C, Memory 60D and an interface I/F60E, connected to each other through a bus B, and is connected to a display unit 62 displaying a variety of information, a variety of instructions, and an input unit 64 for inputting information.

In Memory 60D are stored a variety of programs for controlling the biosensor 10, a variety of data, and a screening program.

The reference data G2 stored in Memory 60D is showed by the amount of variation RU (Resonance Unit, 1 RU=1 pg/mm²) between a reference point evaluated when a running buffer is supplied on the immobilization membrane 50A on which the physiologically active substance D and a signal point evaluated when the candidate molecule A is supplied.

In this way, in the biosensor 10 concerning the embodiment, the sensor stick 40 is conveyed to the measurement unit 56, and when the instruction of measurement initiation from the input unit 64, the measurement processing of binding amount is carried out in the control unit 60. In the measurement processing of binding amount, the solution of an initial candidate molecule of study is supplied to the liquid flow channel 45, the light source 54A, the signal processing unit 54E and a driving system (not shown) is run to obtain the measurement data GI from the measurement region E1 and then to obtain the reference data G2 from the reference region E2. Thereafter, the measurement data G1 are corrected by means of the reference data G2 to calculate the data G3 of binding amount and then to store the data of binding amount for every candidate molecule.

Next, a screening method concerning the embodiment will be set forth.

The embodiment carries out the activity lowering treatment for lowering the physiological activity of the physiologically active substance D, and screens a candidate molecule on the basis of a specified difference occurring prior to and after the activity lowering treatment between the data of binding amount (data of binding amount during activity) in conditions of maintaining the physiological activity of the physiologically active substance D and the data of binding amount (data of binding amount during low activity) in conditions of lowering the physiological activity.

The activity lowering treatment of the physiologically active substance D may enable the lowering of binding activity for a candidate molecule of the physiologically active substance D, and is preferably at least 50% of the binding activity prior to the low activity, more preferably 20% or less, most preferably 10% or less, in order to clarify the difference between the data of binding amount during low activity and the data of binding amount during activity. When the difference is small between the activity prior to the low activity treatment and the activity subsequent to the low activity treatment, the difference of the binding is difficult to detect, and thus a preferred screening result is not obtained.

Examples of the activity lowering treatment can include (1) change of a buffer composition such as pH, and hydrochloric acid concentration, (2) change of the concentration of a surfactant, (3) treatment with an acid, an alkali and an organic solvent, (4) irradiation of light, (5) change of the temperature, (6) addition of a protein modifying agent, and the like. Of these, for (1) to (3), activity lowering treatment can be carried out by means of an operation similar to usual measurement. For (4) and (5), a treating solution needs not be prepared. As such, appropriate activity lowering treatment may be, as required, selected from (1) to (5) above, depending on the kind of the physiologically active substances D.

When a buffer composition is changed, the pH at which the activity of the physiological active substance D is maintained is preferably changed from 0.01 to 7.0, both inclusive, more preferably changed from 0.5 to 4.0. In addition, the concentration of a salt is preferably from 0.2 to 10 mM, more preferably from 0.3 mM to 5 mM. These binding activity lowering treatment solutions can include an acetic acid buffer, a boric acid buffer, and the like, in addition to buffers described above, the salt concentration and pH of which are adjusted.

As a surfactant, a well-known surfactant such as Triton X-100, Tween20, SDS, or the like may be used at 0.1 volume % or more, preferably at 0.5 volume % or more, which is relatively a high concentration.

As an organic solvent, a well-known solvent such as ethanol, DMSO or the like may be used at 10 volume % or more, preferably at 20 volume % or more. The protein modifying agents that can be used include well-known compounds such as phenol, urea, and guanidine hydrochloride.

For the irradiation of light, ultraviolet light may be irradiated at least for 10 minutes. When the temperature is changed, the physiologically active substance D may be treated at temperature of 5° C. or more, preferably at a temperature of 10° C. or more, higher than a suitable temperature of the physiologically active substance D.

When a buffer for treatment is used as an activity lowering treatment solution, an activity lowering treatment solution stock container instead of a buffer solution stock container 18 is mounted, and a solution is supplied by the instruction of activity lowering treatment initiation. This makes it possible to carry out activity lowering treatment similar to the supply of a buffer solution.

This activity lowering treatment may be performed either prior to or subsequent to immobilization of the physiologically active substance D. It is preferable to perform the treatment subsequent to the immobilization of the physiologically active substance D on the immobilization membrane 50A in order not to spoil the immobilization capability of the physiologically active material D. On the other hand, during immobilization of the physiologically active substance D, a substance known to bind to the binding site between the physiologically active material D and a candidate molecule, for example by means of making an inhibitor coexistent, can protect a specific binding site. Thus, when such a substance is made coexistent, activity lowering treatment prior to immobilization or during immobilization can be carried out as well.

Lowering of physiological activity can be confirmed by measuring binding amount of a candidate molecule or the function of the physiologically active material D.

Binding amount between the physiologically active material D and a candidate molecule can be readily confirmed by means of the biosensor by obtaining the data of binding amount G3 of a candidate molecule to the physiologically active material D immobilized on the immobilization membrane 50A.

In this case, the theoretical maximum amount of binding is calculated using the equation below from respective molecules of the physiologically active material D and the candidate molecule.

Theoretical maximum amount of binding=protein immobilization amount×{(molecular weight of a compound that specifically binds)/(molecular weight of protein)}×(number of binding sites of the compound for a protein molecule)

Next, binding amount of the compound to a protein is measured, and the binding ratio is calculated by the equation below. The closer to 100 this ratio is, the more the function and activity of the protein can be considered to be maintained.
Binding ratio=(measurement of binding amount)/(theoretical maximum amount of binding)×100

The methods of measuring the function of the physiologically active material D can include a method of measuring enzyme activity and a method of measuring receptor activity.

The measurement principle of enzyme activity is described in Chapter IV in “Koso No Kisojikkenhou (Basic Experimental Methodology of Enzymes)” (Takekazu Horio and Hitohira Yamashita). Moreover, the methods of detection that can be employed include (1) spectroscopic measuring methods, (2) the fluorescence method, (3) electrode methods, (4) light emitting methods, etc, and also, for example, enzyme immune measuring methods (Shinsei Seikagaku Jikkenkohza Proteins V, Third Edition), methods described in Enzyme Assays Biochemical Experimental Methodology, etc.

For instance, when enzyme activity is measured by the fluorescence method, the reaction of a substrate specific for an enzyme (a substrate in which only its decomposed substance emits fluorescence) can lead to the measurement of enzyme activity by carrying out the fluorescence measurement of the decomposed substance.

When enzyme activity is measured by a spectroscopic measuring method, the utilization of the difference of spectroscopic properties between the substrate and the product can lead to the measurement of enzyme activity by the measurement of its time change and evaluation of initial rate of the enzyme reaction.

When enzyme activity is measured by an electrode method, the method of making use of an automatic titration apparatus enables the measurement of enzyme activity by the electrical detection of the pH change of the sample solution based on an acid or a base generated along with chemical reactions including enzyme reaction.

A light emitting method of measuring enzyme activity involves labeling the antibody or antigen with an enzyme, carrying out antigen-antibody reaction, reacting a chemically emitting substrate (a substrate in which only its decomposed substance emits fluorescence) and then measuring the chemical emission of the decomposed substance, thereby enabling the measurement of enzyme activity.

The measurement of receptor activity involves contacting one species or more of receptors or ligands immobilized on a supporter with a specimen containing the antigen-labeled ligand or receptor, contacting an antigen-labeled ligand or receptor binding to one species or more of the receptors or ligands immobilized on the supporter with an antibody to the antigen (e.g., an antibody labeled with an enzyme for detection) to carrying out antigen-antibody reaction, and then detecting the existence of an antibody bound thereby, which enables the detection of a ligand or receptor in the specimen.

The embodiment immobilizes the physiologically active substance D, and measures the amount of immobilization, and also calculates the values of the function and activity of a protein by utilization of the above method. The amount of immobilization of a protein is not always constant, and thus it is preferred to calculate the activity value per constant protein amount by means of the following equation.
Activity value=protein activity value/protein immobilization value

Extraction of a candidate molecule is preferably carried out through the use of a threshold set in advance.

As such a threshold, the first threshold may be set that is the upper limit value during the selection by use of the data of binding amount during low activity. In this case, a candidate molecule is extracted that shows the data of binding amount during low activity that is the threshold or less. A candidate molecule having the data of binding amount during low activity that is the threshold larger than the first threshold is thought to be a substance that exceeds specifically binding to the physiologically active substance D. This can efficiently remove a candidate molecule non-specifically binding to the physiologically active substance D.

Moreover, in addition to the first threshold, the second threshold value may be set that is the lower limit value during the selection by use of the data of binding amount during activity. In this case, a candidate molecule showing data of binding amount during activity of a threshold amount or more is extracted. A candidate molecule showing data of binding amount during activity of a threshold amount or less than the second threshold is thought to be a substance that has an insufficient amount of binding to the physiologically active substance D. This enables efficient extraction of a candidate molecule that specifically and sufficiently binds to the physiologically active substance D.

Setting of two thresholds like this makes it possible to remove a candidate molecule non-specifically binding to the physiologically active substance D and efficiently extract a candidate molecule that specifically binds in a sufficient amount of binding.

As a threshold, a value obtained as data of binding amount may directly be applied, or a value obtained by dividing a value of the data of binding amount by the molecular weight of a candidate molecule may be used. Because binding amount is proportional to the molecular weight, a value obtained by dividing a value of the data of binding amount by the molecular weight of a candidate molecule is more preferably used from the viewpoints of relative comparison and selection in a substance.

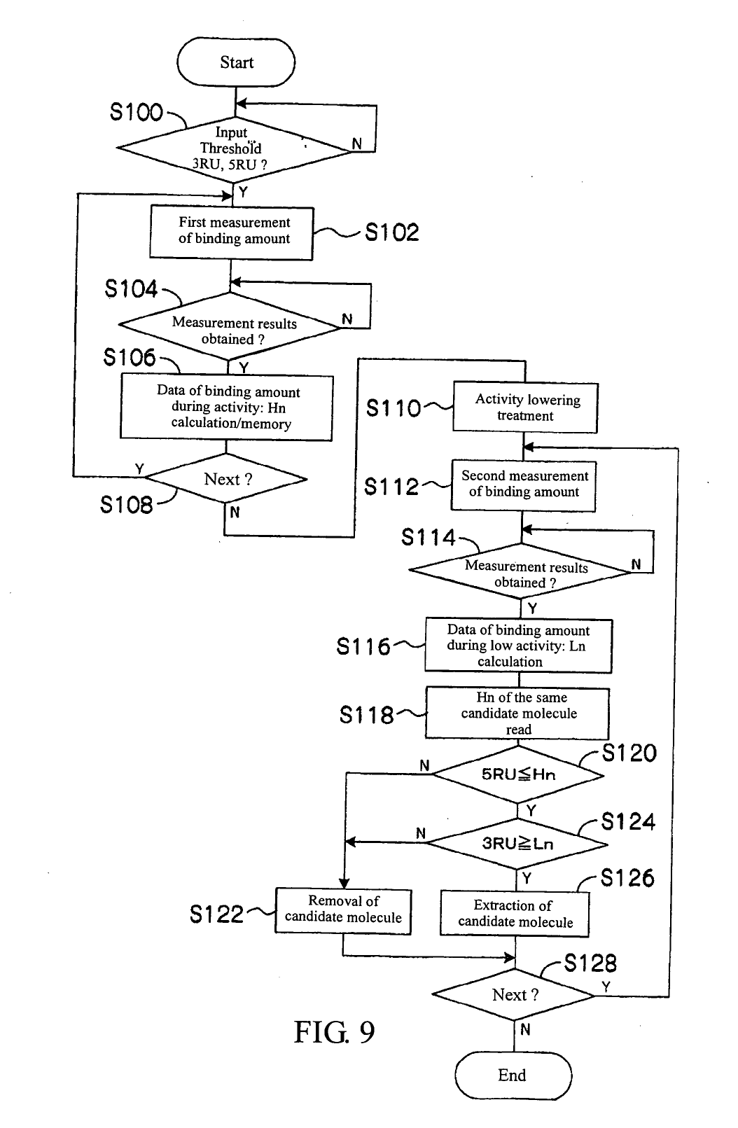

Next, referring to FIG. 9, screening processing by means of the biosensor 10 will be described when the first threshold corresponding to the data of binding amount during activity and the second threshold corresponding to the data of binding amount during low activity are respectively set as “5RU” and “3RU.”

The sensor stick 30 is conveyed to the measurement unit 56. When the instruction of screening initiation from the input unit 64 is input, the screening processing shown in FIG. 9 is carried out in the control unit 60.

In Step S100, whether or not the first threshold “5RU” and the second threshold “3RU” are input is decided. When a user inputs the respective thresholds from the input unit 64, the decision is “Yes” and in Step S102 the first measurement of binding amount is executed, and then in Step S104 the decision is “No” until the measurement result is obtained. The measurement of binding amount is carried out by supplying the first candidate molecule solution of study of a candidate molecule library to the liquid flow channel 45 and by obtaining as measurement results a measurement data G1 from the measurement region E1 and a reference data G2 from the reference region E2.

When the measurement results of the measurement data G1 and the reference data G2 are obtained in Step S104, the decision is “Yes,” and in Step S 106 correction of the measurement data G1 using the reference data G2 leads to the calculation of binding amount during activity data Hn as the binding amount data G3, which is associated to a candidate molecule and stored in Memory 60D. This data of binding amount during activity contains a specific binding component according to the specific binding of a candidate molecule to the physiologically active substance D. Upon completion of the calculation and storage of the data Hn of binding amount during activity, the processing proceeds to Step S108, where whether or not there is a next candidate molecule is decided.

In Step S108, when a next candidate molecule is sensed by a sensor (not shown), the decision is “Yes” and the processing proceeds to Step S102, where the first measurement processing of binding amount is carried out on the next candidate molecule.

If the next candidate molecule is not sensed in Step S108, the decision is “Yes” and the processing proceeds to Step S110, where activity lowering treatment is carried out.

The activity lowering treatment is carried out by supplying the prepared activity lowering treatment solution instead of the running buffer to the liquid flow channel 45 and by reacting the treatment solution with the physiologically active substance D on the immobilization membrane 50A. This treatment lowers a specific binding to a candidate molecule of the physiologically active substance D. After a specified time, the supply of the activity lowering treatment is stopped and the activity lowering treatment is completed by supplying the running buffer to the solution for a specified time, and then the processing proceeds to Step S112.

In Step S112, the second measurement processing of binding amount is carried out on a candidate molecule after obtaining the data of binding amount during activity.

The second measurement processing of binding amount is carried out as in the first measurement of binding amount with the exception that the physiologically active substance D used is that that has been subjected to activity lowering treatment. In Step S114, the decision is then “No” until the measurement results of the measurement data G1 and the reference data G2 are obtained. When the measurement results are obtained, the decision is “Yes” and the processing proceeds to Step S116, where the binding amount during low activity data Ln as the binding amount data G3 is calculated as described above. In the data Ln of binding amount during low activity, the binding activity of the physiologically active substance D is lowered due to activity lowering treatment, and so theoretically the amount of specific binding component contained in the data becomes smaller than the specific binding component contained in the data value of binding amount during activity.

When the data Ln of binding amount during low activity is calculated, in Step S118 the data Hn of binding amount during activity corresponding to that of the same candidate molecule is read from Memory 60D, and in Step S120 whether or not the data Hn of binding amount during activity is at the first threshold 5RU is decided.

If the data Hn of binding amount during activity is smaller than 5RU, the decision is “No” and the processing proceeds to Step S122. On the other hand, if the data Hn of binding amount during activity is 5RU or more, the decision is “Yes” and the processing proceeds to Step S124.

In Step S124, whether or not the data Ln of binding amount during low activity is at the second threshold 3RU or less is decided, and when the data Ln is larger than 3RU, the decision is “No” and the processing proceeds to Step S122. In Step S122, the candidate molecule being a target is decided to be a candidate molecule that non-specifically binds to the physiologically active substance D and is removed from extraction targets, and then proceeding proceeds to Step S128.

On the other hand, when the data Ln of binding amount during low activity is 3RU or less, the decision is “Yes” and the processing proceeds to Step S126. In Step S126, the candidate molecule being a target is decided to be a candidate molecule that should be extracted, and then proceeding proceeds to Step S128.

In Step S128, whether or not a next candidate molecule is present is decided, and if a sensor (not shown) senses a next candidate molecule, the decision is “Yes” and the processing proceeds to Step S112, where the second measurement processing of binding amount is carried out on the next candidate molecule.

On the other hand, if the sensor does not sense a next candidate molecule, the decision is “No” and then in the extraction step the candidate molecule(s) extracted is/are displayed on the display unit 62 and also Memory 60D is cleared, and then the processing is completed.

Execution of screening processing of the embodiment can remove from extraction targets a candidate molecule that is high in non-specific binding and indicates the data of binding amount during low activity that exceeds 3RU and extract a candidate molecule(s) that exhibit(s) low non-specific binding and high binding activity of 5RU or more. This makes it possible to simply and easily and efficiently carry out the screening of a substance specifically binding to the physiologically active substance D on a large number of candidate molecules.

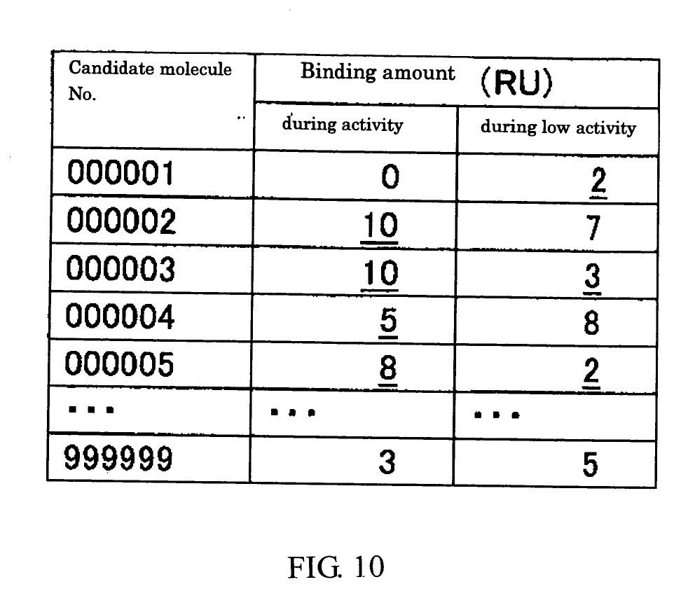

FIG. 10 shows an example of a list showing the results of binding amount memorized per candidate molecule (data Hn of binding amount during activity and the data Ln of binding amount during low activity).

The implementation of screening on such a candidate molecule through the use of the first threshold 3RU and the second threshold 5RU extracts the two candidate molecules Nos. 000003 and 000005. In particular, the implementation of screening enables the removal of a candidate molecule that exhibits a small difference between the data of binding amount during low activity and the data of binding amount during activity, such as the candidate molecule No. 000002, and is thought to tend to non-specifically bind, from extraction targets.

Although the embodiment extracts a candidate molecule using the first and the second thresholds, the embodiment does not limit to this.

For instance, a candidate molecule may be extracted by using the threshold of 1 corresponding to the difference between the data Hn of binding amount during activity and the data Ln of binding amount during low activity, or using the threshold corresponding this difference and the first threshold used in the embodiment or the second threshold in combination. It is possible for a user to select, as required, a threshold or a combination of thresholds, depending on the extent of reducing the number of candidate molecules or target molecules, or screening methods.

### Second Embodiment

Next, the second embodiment of the invention will be set forth.

The description of the configuration of the biosensor 10 will not be repeated here since the biosensor 10 used in the first embodiment is directly applicable as a screening device applicable to the second embodiment.

The second embodiment of the invention entails extracting a candidate molecule by comparing the data of binding amount during activity with the first threshold, carrying out the second measurement processing of binding amount only on a candidate molecule extracted, and subsequently extracting a candidate molecule specifically binding to a physiologically active substance D.

Now, the first threshold is used for extracting a candidate molecule having a large amount of binding to the physiologically active substance D and also extracting a candidate molecule that is a target of the measurement of binding amount for obtaining the data of binding amount during low activity. This enables the reduction of the number of measurement targets of the second measurement of binding amount, and thus enables the screening of a candidate molecule for a shorter time.

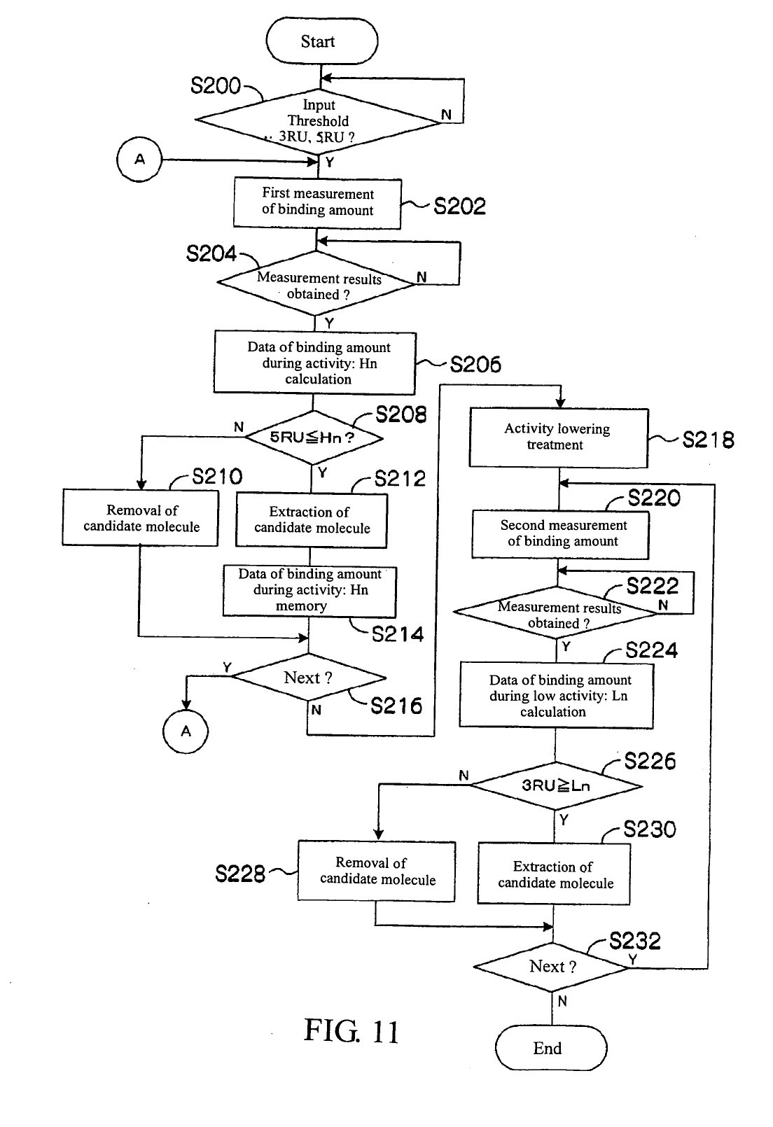

Referring to FIG. 11, screening processing by means of the biosensor 10 will be set forth when the first threshold corresponding to data of binding amount during low activity and the second threshold corresponding to data of binding amount during activity are respectively set as “5RU” and “3RU” hereinafter.

A sensor stick 30 is conveyed to a measurement unit 56. When the instruction of screening initiation from an input unit 64 is input, the screening processing shown in FIG. 11 is carried out in the control unit 60.

In Step S200, whether or not the first threshold “3RU” and the second threshold “5RU” are input is decided. When a user inputs the respective thresholds from the input unit 64, the decision is “Yes” and the processing proceeds to Step S202. Thereafter, the first measurement of binding amount is executed and in Step S204 the decision is “No” until the measurement results of data G1 and a reference data G2 are obtained.

When the measurement results of the data G1 and the reference data G2 are obtained in Step S204, the decision is “Yes” and the processing proceeds to Step S206, where correction of the measurement data G1 using the reference data G2 leads to the calculation of binding amount during activity data Hn as the binding amount data G3, and then the processing proceeds to Step S208.

In Step S208, as the first extraction step, whether or not the data Hn of binding amount during activity is at the first threshold 5RU or more is decided. When the data Hn is smaller than 5RU, the decision is “No” and the processing proceeds to Step S210, where the candidate molecule being a target is removed from an extraction target, and then the processing proceeds to Step S216.

When the data Hn of binding amount during activity is 5RU or more, the decision is “Yes” and the processing proceeds to Step S212. In Step S212, the candidate molecule being a target is extracted, and in Step S214 the data Hn of binding amount during activity is associated to the extracted candidate and stored in Memory 60D. After the data Hn of binding amount during activity is stored, the processing proceeds to Step 216.

In Step S216, whether or not the next candidate molecule is present is decided, and when the next candidate molecule is sensed, the decision is “Yes” and the processing proceeds to Step S202, where the first measurement of binding amount is carried out on the next candidate molecule.

If the next candidate molecule is not sensed in Step S216, the decision is “No” and the processing proceeds to S218, where specified activity lowering treatment is carried out and then the activity lowering treatment is completed. Thereafter, in Step S220 the second processing of binding amount is carried out. In this second measurement processing of binding amount, only a candidate molecule extracted in the first extraction step is selected and the processing is carried out.

When the second measurement processing of binding amount is carried out in Step S220, in Step S222 whether or not the measurement result is obtained is decided. Thereafter, if the measurement result is obtained, the decision is “Yes” and in Step S224 data Ln of binding amount during low activity as data G3 of binding amount is calculated.

After the data Ln of binding amount during low activity is calculated, in Step S226, as a second extraction step, whether or not the data Ln of binding amount during low activity is at the second threshold 3RU or less is decided.

When the data Ln of binding amount during low activity is larger than 3RU, the decision is “No” and the processing proceeds to Step S228, where the candidate is determined to be a candidate molecule capable of non-specifically binding to the physiologically active substance D and is removed from extraction targets, and then the processing proceeds to Step S232.

On the other hand, in Step S226, when the data Ln of binding amount during low activity is 3RU or less, the decision is “Yes” and the processing proceeds to Step S230, where the candidate molecule being a target is determined to be a candidate molecule that binds to the physiologically active substance D and should be extracted, and then the processing proceeds to Step S232.

In Step S232, whether or not the next candidate molecule is present is decided, and if a next candidate molecule is sensed, the decision is “Yes” and the processing proceeds to Step S220, where the second measurement processing of binding amount is carried out on the next extraction candidate molecule.

On the other hand, if the sensor does not sense a next candidate molecule, the decision is “No” and then in the second extraction step the candidate molecule(s) extracted is/are displayed on the display unit 62 and also Memory 60D is cleared, and then the processing is completed.

Execution of screening processing of the embodiment can remove a candidate molecule that is high in non-specific binding and shows the data of binding amount during low activity that exceeds 3RU and extract a candidate molecule that exhibits low non-specific binding and high binding activity of 5RU or more. This makes it possible to simply and easily and efficiently carry out the screening of a substance specifically binding to the physiologically active substance D on a large number of candidate molecules.

In addition, candidate molecules to be the target of the second measurement processing of binding amount are a small number of candidate molecules that are extracted by reduction in the extraction step, so the frequency of the second measurement processing of binding amount can be decreased, which can efficiently screen candidate molecules specifically binding to the physiologically active substance D for a shorter time.

In the embodiment of the invention, although the first threshold for the data of binding of the amount of low activity is set to be 3RU, and the second threshold for the data of binding of the amount of activity is set to be 5RU, the thresholds are not limited thereto. The thresholds may be the same value, or different values. A user can set, as required, the value, depending on the properties of a candidate molecule or the physiologically active substance D, or the extent of specific binding to be required.

Moreover, in the embodiment of the invention, although a candidate molecule extracted as a substance specifically binding to the physiologically active substance D is displayed on the display unit 62 and Memory 62D is cleared, the embodiment is not limited thereto. The data of binding amount of all the candidate molecules that are subjected to the measurement processing of binding amount are made to be contrasted with candidate molecules and then may be stored as data.

Furthermore, in the embodiment of the invention, screening processing is carried out, including the measurement processing of binding amount; however, only the extraction step may be subjected to screening processing. In this case, a list of the data of binding amount is made by corresponding the data of binding amount obtained before and after activity lowering treatment by means of measurement processing of binding amount to all the candidate molecules, and screening processing can be initiated by reading this list of the data of binding amount.

Additionally, in the embodiment of the invention, the physiologically active substance D is immobilized on an immobilization membrane 50A and then activity lowering treatment is carried out thereon, but the embodiment is not limited thereto. Activity lowering treatment may be performed prior to immobilization of the physiologically active substance D on the immobilization membrane 50A, depending on the kinds of physiologically active substances D and activity lowering treatments. For the comparison of the data of binding amount of the case of no implementation of activity lowering treatment and the case of implementation of activity lowering treatment, the amounts of physiologically active substances D are preferably almost the same in the both cases, and thus activity lowering treatment is preferably carried out after immobilization.

In the embodiment, the data of binding amount prior to activity lowering treatment based on binding ability is set to be the data of binding amount during activity and the data of binding amount after processing is set to be the data of binding amount during low activity, but the embodiment is not limited thereto. The data of binding amount during activity may be data of no activity lowering treatment, or may be data processed to some extent. In this case, for a substance maintaining activity having at least 40%, preferably 60% or more, more preferably 80% or more is obtained the data of binding amount during activity.

In the embodiment of the invention, the physiologically active substance D is used as a target molecule immobilized on the immobilization membrane 50A, and a candidate molecule supplied to the liquid flow channel 45 is set to be an organic or inorganic molecule capable of binding to the physiologically active substance D, but the embodiment is not limited thereto. The physiologically active substance D is set to be a candidate molecule and an organic or inorganic molecule capable of binding thereto may also be set to be a target molecule.

Moreover, in the embodiment of the invention, a surface plasmon sensor is described as the biosensor, but the biosensor is not limited to a surface plasmon sensor. For example, a reference data obtained in a reference region in the case of using any biosensor, such as a Quartz Cystal Microbalance (QCM) measuring technique, an optical measuring technique using a functional surface from colloidal particles of gold to superfine particles, or the like, is made to be a data base for storage, and a measured measurement data can also be corrected using the reference data thus stored.

In addition, other biosensors making use of the attenuated total reflection can include the leaky mode sensor. The leaky mode sensor includes a dielectric, and a thin membrane consisting of a clad layer and a light wave guide layer provided on the dielectric sequentially. One face of this thin layer is the sensor face and the other face is the light incidence face. When light is put in to the light incident face so as to satisfy the total reflection conditions, part of the light passes through the above clad layer and is taken in the above light incidence face. Thereafter, when the guide wave mode is excited in this light wave guide layer, the reflected light at the above light incident face is greatly attenuated. The incident angle by which the guide wave mode is excited varies depending on the refraction index of a medium over the sensor face, as with the surface plasmon resonance angle. The detection of this refracted light can lead to the measurement of the reaction on the above sensor face.

### EXAMPLE

Next, the fabrication of the immobilization membrane 50A on the dielectric block 42 described in the above embodiment, the immobilization of a physiologically active substance D on the immobilization membrane 50A fabricated, and the binding of a candidate molecule to the physiologically active substance D immobilized will be set forth.

### Example 1

(1) Fabrication of a Measuring Chip

A dielectric block on which 50 nm of a metal membrane of gold had been deposited was treated using a Model-208UV-ozone cleaning system (TECHNOVISION INC.) for 30 minutes, and then a 1.0 mM solution of 16-hydroxy-1-hexadecanethiol in ethanol water (80/20: volume ratio) was dropped so as to be contacted with the metal membrane and the surface treatment was carried out at 25° C. for one hour. Thereafter, the metal membrane was washed with ethanol 5 times, a mixture solvent of ethano1water once, and water 5 times.

Next, the surface coated with 16-hydroxy-1-hexadecanethiol was contacted with a 10 weight (mass) % epichlorohydrin solution (1:1 mixture solution of 0.4 M sodium hydroxide and diethylene glycol dimethyl ether) and the resulting material was reacted for 4 hours in a shaking incubator at 25° C. Subsequently, the surface was washed with ethanol twice and water 5 times. Next, to 40.5 ml of a 25 weight % aqueous solution of dextran (T500, Pharmacia) was added 4.5 ml of 1M sodium hydroxide, and the resulting solution was contacted with the epichlorohydrin treated surface. Thereafter, the resulting material was incubated in a shaking incubator at 25° C. for 20 hours. The surface was washed with water at 50° C. 10 times. Subsequently, a mixture solution of 3.5 g of bromoacetic acid in 27 g of 2M sodium hydroxide solution was contacted with the above dextran treated surface, and then the resulting material was incubated in a shaking incubator at 25° C. for 16 hours. The surface was washed with water, and then similar bromoacetic acid treatment was further carried out once.

(2) Immobilization of Protein

In the following procedure, p38MAP kinase (#559324, available from CALBIOCHEM Inc.) was immobilized to the hydrogel coated measuring chip fabricated in (1).

First, a protein solution containing 200 μg/ml of p38MAP kinase and 10 μM of an inhibitor listed in Table 1 is prepared. The composition of the running buffer is as follows: 50 mM phosphoric acid buffer (pH 7.2)/150 mM NaCl/3.4 mM EDTA.

First, onto the hydrogel coated chip was dropped the running buffer to draw a baseline. Next, a mixture solution of 1-ethyl-2,3-dimethylaminopropyl carbodiimide (400 mM) and N-hydroxysuccinimide (100 mM) is dropped thereonto, and the resulting material is allowed to stand for 15 minutes, and then the chip is washed with the running buffer. Thereafter, the protein solution prepared is dropped thereonto, and then the resulting material is allowed to stand for 20 minutes and the chip was washed with a PBS buffer (pH 7.4). Further, an ethanolamine/HCl solution (1M, pH 8.5) was dropped thereonto and the resulting material is allowed to stand for 15 minutes and then the chip was washed with the running buffer 3 times. The value in the change of the signal from the first baseline was set to be the amount of immobilization of p38 kinase (RU).

The results are listed in Table 1. Table 1 shows that as the pH of the immobilization buffer is increased, the amount of immobilization is decreased, so the immobilization cannot be done at a pH of 6.0.

TABLE 1

| | Protein Immobilization Conditions | | Protein |
|---|---|---|---|
| Sample | Immobilization | | Immobilization |
| No. | Buffer | Inhibitor | Amount (RU) |
| A | Acetic acid buffer, | Compound 1, | 6100 |
| | pH 5.00 | 10 μM | |
| B | Acetic acid buffer, | None | 5000 |
| | pH 5.50 | | |
| C | Acetic acid buffer, | Compound 1, | 5800 |
| | pH 5.50 | 10 μM | |
| D | Acetic acid buffer, | Compound 1, | 3500 |
| | pH 5.75 | 10 μM | |
| E | Acetic acid buffer, | Compound 1, | 0 |
| | pH 6.00 | 10 μM | |

(3) Binding Measurement of Compounds

A measuring chip (excepting Sample E) on which the protein had been immobilized was fixed to a commercially available surface plasmon response-based resonance measuring device, and the binding measurements of Compounds 1 to 3 were carried out in the following manner. Compound 1 is a compound that is well-known to inhibit the activity of p38MAP kinase. On the other hand, Compounds 2 and 3 are compounds that do not inhibit the activity of p38MAP kinase.

The running buffer having the following composition was prepared: 50 mM phosphoric acid buffer (pH 7.2)/150 mM NaCl/3.4 mM EDTA/DMSO 5 volume %.

Compounds 1 to 3 listed in Table 2 each were dissolved in the running buffer such that the concentrations of the compounds were 10 μM. Thereafter, the running buffer was dropped onto the hydrogel coated chip to draw a baseline. Subsequently, the compound solution prepared was dropped onto the chip and the resulting material was allowed to stand for 3 minutes and then the amount in signal change was set to be binding amount (RU) of each compound. Lastly, the chip was washed with the running buffer.

The results are listed in Table 2. The theoretical maximum amount of binding refers to binding amount when the protein binds to the compound in the ratio 1:1. The molecular weight of p38MAP kinase is 41,000 and that of Compound 1 is 377.

Theoretical maximum amount of binding=protein immobilization amount x (molecular weight of a compound/molecular weight) of the protein The ratio of binding of Compound 1 is calculated by means of the equation below. Compound 1 is known to specifically bind to the activity site of p38MAP kinase, so the closer to 100 the ratio is, the better the activity of the protein is thought to be maintained.
Binding ratio=(measurement of binding amount/theoretical maximum amount of binding)×100

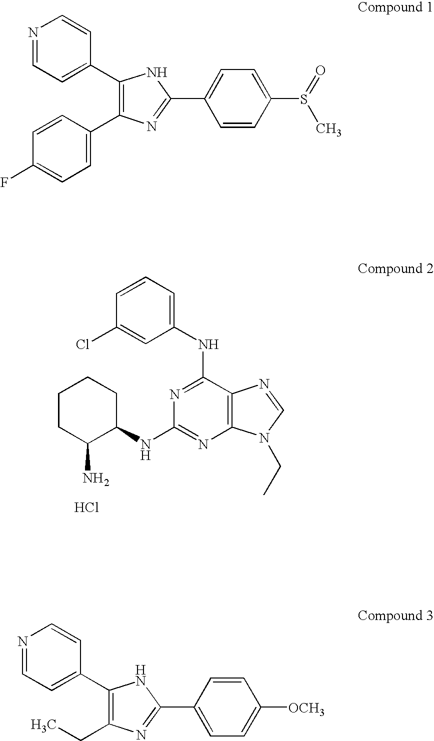 TABLE 2

| | Compound 1 | | | | |
|---|---|---|---|---|---|
| | Theoretical | | | Compound 2 | Compound 3 |
| | maximum | Measurement | | Measurement | Measurement |
| | amount of | of binding | Binding ratio | of binding | of binding |
| Sample No. | binding (RU) | amount (RU) | (%) | amount (RU) | amount (RU) |
| A | 55.5 | 11.0 | 19.8 | 49.1 | 1.3 |
| B | 45.5 | 25.1 | 55.2 | 42.5 | 1.1 |
| C | 52.8 | 34.5 | 65.3 | 42.0 | 1.1 |
| D | 31.9 | 24.0 | 75.2 | 21.5 | 0.8 |

Table 2 shows that the higher the immobilization pH, the lower the activity and that the presence of the inhibitor during immobilization decreases the activity lowering.

In Compound 1 specifically binding to p38MAP kinase, as the activity of p38MAP kinase decreases, binding amount decreases. On the other hand, binding amount of Compound 2 does not decrease even though the activity of p38MAP kinase decreases. Compound 2 is estimated to non-specifically bind to p38MAP kinase. Compound 3 does not binds in any of the samples.

Now, when only the case of Sample D is considered and a screening is decided, Compounds 1 and 2 have respectively 24.0 RU and 21.5 RU, that is, there is almost no difference between them, and thus both are determined to be an extraction target (hit compound). When data are obtained for a low state of protein activity, as with Sample A, Compound 2 can be determined to be a compound that non-specifically adsorbs.

### Example 2

After the protein is immobilized, an example of the methods of decreasing the activity of the protein is shown below.

p38MAP kinase was immobilized in the presence of Compound 1 as in the case of Sample D of Example 1, and then the activity lowering treatment indicated in Table 3 was carried out. Further, the binding of Compound 1 was measured in the same manner as in Example 1 to calculate the binding ratio. The results are listed in Table 3. As shown in Table 3, this simple and easy processing can lower the binding activity of p38MAP kinase.

TABLE 3

| | | | | | Compound 1 | | | |
|---|---|---|---|---|---|---|---|---|
| | Protein immobilization | | | Amount of | Theoretical | Measurement | | Compound 2 |
| | conditions | | | protein | maximum | of binding | | Measurement of |
| Sample | Kind of | | Activity lowering | immobilization | binding amount | amount | Binding | binding amount |
| No. | buffers | Inhibitor | treatment | (RU) | (RU) | (RU) | ratio (%) | (RU) |
| D | Acetic acid | Compound 1, 10 μM | None | 3500 | 31.9 | 24.0 | 75.2 | 21.5 |
| | buffer, pH 5.75 | | | | | | | |
| F | Acetic acid | Compound 1, 10 μM | Acetic acid | 3500 | 31.9 | 4.8 | 15.0 | 50.1 |
| | buffer, pH 5.75 | | buffer, pH 4.50, | | | | | |
| | | | for 30 min. | | | | | |

Thereafter, setting of the first threshold to be, for example, 20RU and of the second threshold to be 5RU enables the extraction of Compound 1 showing the data of the amounts of binding of 20RU or more and 5RU or less. On the other hand, Compound 2 is extracted based on the first threshold, but is removed based on the second threshold, by means of the same screening processing.

Hence, in the case where a candidate molecule is screened that specifically binds to p38MAP kinase, the obtaining of data of binding amount during low activity by implementation of activity lowering treatment using acetic acid buffer makes it possible to simply and easily separate a candidate molecule specifically binding to p38MAP kinase from a candidate molecule non-specifically binding to p38MAP kinase.

In this manner, a candidate molecule having a large difference between data of binding amount prior to activity lowering treatment and data of binding amount subsequent to activity lowering treatment can efficiently and simply and easily be extracted as a substance specifically binding to p38MAP kinase.

According to the invention a candidate molecule capable of non-specifically binding to a target molecule can be removed efficiently, thereby a candidate molecule capable of specifically binding to the target molecule can be extracted simply and easily.

The invention also includes the following embodiments.

<1> A screening method for selecting a particular candidate molecule by use of data of binding amount between a target molecule and the candidate molecule, the data being obtained by supplying the candidate molecule of study to a measurement region composed of an immobilization membrane on which the target molecule is immobilized, the screening method comprising: obtaining the data of binding amount during activity by supplying the candidate molecule to a measurement region in which the target molecule not subjected to activity lowering treatment that lowers physiological activity is immobilized, obtaining the data of binding amount during low activity by supplying the candidate molecule to a measurement region in which the target molecule that is subjected to activity lowering treatment that lowers physiological activity is immobilized, and extracting a candidate molecule having a specified difference between the data of binding amount during the activity and the data of binding amount during the low activity.

<2> The screening method of item <1>, wherein the extracting a candidate molecule includes extracting a candidate molecule showing data of binding amount during low activity of a threshold amount set in advance or less, or a candidate molecule showing data of binding amount during low activity of less than a threshold amount set in advance.

<3> The screening method of item <2>, wherein the extracting a candidate molecule includes extracting a candidate molecule showing data of binding amount during activity having a threshold amount set in advance or more, or a candidate molecule showing data of binding amount during activity exceeding a threshold amount set in advance.

<4> The screening method of any one of items <1> to <3>, further comprising: carrying out activity lowering treatment on the target molecule subsequent to the obtaining of data of binding amount during activity.

<5> The screening method of any one of items <1> to <4>, wherein: the activity lowering treatment on the target molecule is carried out after the target molecule is immobilized on an immobilization membrane.

<6> The screening method of any one of items <1> to <5>, wherein: the immobilization membrane is formed on one side of a metal membrane and not formed on the other side, and the obtaining data of binding amount during activity and the obtaining data of binding amount during low activity are carried out by a process including using attenuated total reflection generated by irradiating a light beam to the opposite side of the metal membrane to that on which the immobilization membrane is formed.

<7> The screening method of item <6>, wherein: obtaining the data of binding amount during activity and obtaining the data of binding amount during low activity is carried out by a process including supplying the candidate molecule to the metal membrane on which the immobilization membrane is formed on which the target molecule is not immobilized, and by setting as a reference data the amount of attenuated total reflection generated by irradiating the light beam to the opposite side of the metal membrane to that on which the immobilization membrane on which the target molecule is not immobilized is formed.

<8> A screening program for selecting a particular candidate molecule by use of data of binding amount between a target molecule and a candidate molecule, the data being obtained by supplying the candidate molecule of study to a measurement region composed of an immobilization membrane on which the target molecule is immobilized, the screening program causing a computer to execute operations, the screening program operations comprising: calculating data of binding amount during activity, based on binding amount between the target molecule and the candidate molecule supplied to a measurement region in which the target molecule not subjected to activity lowering treatment that lowers physiological activity is immobilized, calculating data of binding amount during low activity, based on binding amount between the target molecule and the candidate molecule supplied to a measurement region in which the target molecule that is subjected to activity lowering treatment that lowers physiological activity is immobilized, and extracting a candidate molecule showing a specified difference between the data of binding amount during the activity and the data of binding amount during the low activity.

<9> The screening program of item <8>, further comprising: executing activity lowering treatment of supplying to the target molecule an activity lowering treatment solution for lowering the physiological activity of the target molecule after calculating the data of binding amount during activity, and instructing proceeding to the calculating the data of binding amount during the low activity after the completion of the activity lowering treatment.

<10> The screening program of items <8> or <9>, wherein the extracting includes comparing a threshold amount that has been input for selecting a candidate molecule using the data of binding amount during the low activity with data of binding amount during low activity, and extracting a candidate molecule showing data of binding amount during low activity of the threshold amount or less, or. extracting a candidate molecule showing data of binding amount during low activity of less than the threshold amount.

<11> The screening program of item <10>, wherein the extracting includes comparing a threshold amount input for selecting a candidate molecule using the data of binding amount during activity with data of binding amount during activity, and extracting a candidate molecule showing the data of binding amount during activity of the threshold amount or more, or extracting a candidate molecule showing the data of binding amount during activity exceeding the threshold amount.

<12> The screening program of any one of items <8> to <11>, wherein: the immobilization membrane is formed on one side of a metal membrane and not formed on the other side, and obtaining the data of binding amount during activity and obtaining the data of binding amount during low activity is carried out by a process including using attenuated total reflection generated by irradiating a light beam to the opposite side of the metal membrane to that on which the immobilization membrane is formed.

<13> The screening program of item 12, wherein: obtaining the data of binding amount during activity and obtaining the data of binding amount during low activity are carried out by a process including supplying the candidate molecule to the metal membrane on which the immobilization membrane is formed on which the target molecule is not immobilized, and based on reference data obtained from the amount of attenuated total reflection generated by irradiating the light beam to the opposite side of the metal membrane to that on which the immobilization membrane on which the target molecule is not immobilized is formed.

<14> A screening device for selecting a particular candidate molecule by use of data of binding amount between a target molecule and a candidate molecule, the data being obtained by supplying the candidate molecule of study to a measurement region composed of an immobilization membrane on which the target molecule is immobilized, the screening device comprising: a measuring unit that obtains the data of binding amount by supplying a candidate molecule to the measurement region; an activity lowering treatment mechanism that lowers the physiological activity of the target molecule by supplying an activity lowering treatment solution to the measurement region; a memory that stores for every candidate molecule the data that is obtained from the measuring unit of the active binding amount of the candidate molecule to the target molecule not subjected to the activity lowering treatment, and stores for every candidate molecule data that is obtained from the measuring unit at low activity of the binding amount of an candidate molecule to a low activity target molecule after the activity lowering treatment; and extracting unit that extracts a candidate molecule having a specified difference between the data of active binding amount stored in the memory and the low activity measurement data.

<15> The screening device of item 14, wherein the extracting unit extracts a candidate molecule showing data of binding amount during low activity of a threshold that has been input or less, or extracts a candidate molecule showing data of binding amount during low activity of less than the threshold that has been input.

<16> The screening device of item 15, wherein the extracting unit extracts a candidate molecule showing data of binding amount during activity of a threshold that has been input or more, or extracts a candidate molecule showing data of binding amount during activity exceeding the threshold.

<17> The screening device of any one of items 14 to 16, wherein: the immobilization membrane is formed on one side of a metal membrane and is not formed on the other side, and obtaining the data of binding amount during activity and obtaining the data of binding amount during low activity are carried out by a process including using attenuated total reflection generated by irradiating a light beam to the opposite side of the metal membrane to that on which the immobilization membrane is formed.

<18> The screening device of item 17, wherein: obtaining the data of binding amount during activity and obtaining the data of binding amount during low activity are carried out by a process including supplying the candidate molecule to a metal membrane on which is formed the immobilization membrane on which the target molecule is not immobilized, the data being based on reference data obtained from the amount of attenuated total reflection generated by irradiating the light beam to the opposite side of the metal membrane to that on which the immobilization membrane on which the target molecule is not immobilized is formed.

All publications, patent applications, and technical standards mentioned in this specification are herein incorporated by reference to the same extent as if such individual publication, patent application, or technical standard was specifically and individually indicated to be incorporated by reference.

It will be obvious to those having skill in the art that many changes may be made in the above-described details of the preferred embodiments of the present invention. The scope of the invention, therefore, should be determined by the following claims.

What is claimed is:

1. A screening method for selecting a particular candidate molecule by use of data of binding amount between a target molecule and the candidate molecule, the data being obtained by supplying the candidate molecule of study to a measurement region composed of an immobilization membrane on which the target molecule is immobilized, the screening method comprising:
obtaining the data of binding amount during activity by supplying the candidate molecule to a measurement region in which the target molecule not subjected to activity lowering treatment that lowers physiological activity is immobilized,
obtaining the data of binding amount during low activity by supplying the candidate molecule to a measurement region in which the target molecule that is subjected to activity lowering treatment that lowers physiological activity is immobilized, and
extracting a candidate molecule having a specified difference between the data of binding amount during the activity and the data of binding amount during the low activity.

2. The screening method of claim 1, wherein
the extracting a candidate molecule includes extracting a candidate molecule showing data of binding amount during low activity of a threshold amount set in advance or less, or a candidate molecule showing data of binding amount during low activity of less than a threshold amount set in advance.

3. The screening method of claim 2, wherein
the extracting a candidate molecule includes extracting a candidate molecule showing data of binding amount during activity having a threshold amount set in advance or more, or a candidate molecule showing data of binding amount during activity exceeding a threshold amount set in advance.

4. The screening method of claim 1, further comprising:
carrying out activity lowering treatment on the target molecule subsequent to the obtaining of data of binding amount during activity.

5. The screening method of claim 1, wherein:
the activity lowering treatment on the target molecule is carried out after the target molecule is immobilized on an immobilization membrane.

6. The screening method of claims 1, wherein:
the immobilization membrane is formed on one side of a metal membrane and not formed on the other side, and
the obtaining data of binding amount during activity and the obtaining data of binding amount during low activity are carried out by a process including using attenuated total reflection generated by irradiating a light beam to the opposite side of the metal membrane to that on which the immobilization membrane is formed.

7. The screening method of claim 6, wherein:
obtaining the data of binding amount during activity and obtaining the data of binding amount during low activity are carried out by a process including supplying the candidate molecule to the metal membrane on which the immobilization membrane is formed on which the target molecule is not immobilized, and by setting as a reference data the amount of attenuated total reflection generated by irradiating the light beam to the opposite side of the metal membrane to that on which the immobilization membrane on which the target molecule is not immobilized is formed.

8. A screening program for selecting a particular candidate molecule by use of data of binding amount between a target molecule and a candidate molecule, the data being obtained by supplying the candidate molecule of study to a measurement region composed of an immobilization membrane on which the target molecule is immobilized, the screening program causing a computer to execute operations, the screening program operations comprising:
calculating data of binding amount during activity, based on binding amount between the target molecule and the candidate molecule supplied to a measurement region in which the target molecule not subjected to activity lowering treatment that lowers physiological activity is immobilized,
calculating data of binding amount during low activity, based on binding amount between the target molecule and the candidate molecule supplied to a measurement region in which the target molecule that is subjected to activity lowering treatment that lowers physiological activity is immobilized, and
extracting a candidate molecule showing a specified difference between the data of binding amount during the activity and the data of binding amount during the low activity.

9. The screening program of claim 8, further comprising:
executing activity lowering treatment of supplying to the target molecule an activity lowering treatment solution for lowering the physiological activity of the target molecule after calculating the data of binding amount during activity, and
instructing proceeding to the calculating the data of binding amount during the low activity after the completion of the activity lowering treatment.

10. The screening program of claim 8, wherein
the extracting includes comparing a threshold amount that has been input for selecting a candidate molecule using the data of binding amount during the low activity with data of binding amount during low activity, and extracting a candidate molecule showing data of binding amount during low activity of the threshold amount or less, or extracting a candidate molecule showing data of binding amount during low activity of less than the threshold amount.

11. The screening program of claim 9, wherein
the extracting includes comparing a threshold amount that has been input for selecting a candidate molecule using the data of binding amount during the low activity with data of binding amount during low activity, and extracting a candidate molecule showing data of binding amount during low activity of the threshold amount or less, or extracting a candidate molecule showing data of binding amount during low activity of less than the threshold amount.

12. The screening program of claim 10, wherein
the extracting includes comparing a threshold amount input for selecting a candidate molecule using the data of binding amount during activity with data of binding amount during activity, and extracting a candidate molecule showing the data of binding amount during activity of the threshold amount or more, or extracting a candidate molecule showing the data of binding amount during activity exceeding the threshold amount.

13. The screening program of claim 11, wherein
the extracting includes comparing a threshold amount input for selecting a candidate molecule using the data of binding amount during activity with data of binding amount during activity, and extracting a candidate molecule showing the data of binding amount during activity of the threshold amount or more, or extracting a candidate molecule showing the data of binding amount during activity exceeding the threshold amount.

14. The screening program of claims 8, wherein:
the immobilization membrane is formed on one side of a metal membrane and not formed on the other side, and
obtaining the data of binding amount during the activity and obtaining the data of binding amount during the low activity is carried out by a process including using attenuated total reflection generated by irradiating a light beam to the opposite side of the metal membrane to that on which the immobilization membrane is formed.

15. The screening program of claim 14, wherein:
obtaining the data of binding amount during activity and obtaining the data of binding amount during low activity are carried out by a process including supplying the candidate molecule to the metal membrane on which the immobilization membrane is formed on which the target molecule is not immobilized, and based on reference data obtained from the amount of attenuated total reflection generated by irradiating the light beam to the opposite side of the metal membrane to that on which the immobilization membrane on which the target molecule is not immobilized is formed.

16. A screening device for selecting a particular candidate molecule by use of data of binding amount between a target molecule and a candidate molecule, the data being obtained by supplying the candidate molecule of study to a measurement region composed of an immobilization membrane on which the target molecule is immobilized, the screening device comprising:
a measuring unit that obtains the data of binding amount by supplying a candidate molecule to the measurement region;
an activity lowering treatment mechanism that lowers the physiological activity of the target molecule by supplying an activity lowering treatment solution to the measurement region;
a memory that stores for every candidate molecule the data that is obtained from the measuring unit of the active binding amount of the candidate molecule to the target molecule not subjected to the activity lowering treatment, and stores for every candidate molecule data that is obtained from the measuring unit at low activity of the binding amount of an candidate molecule to a low activity target molecule after the activity lowering treatment; and
extracting unit that extracts a candidate molecule having a specified difference between the data of active binding amount stored in the memory and the low activity measurement data.

17. The screening device of claim 16, wherein
the extracting unit extracts a candidate molecule showing data of binding amount during low activity of a threshold that has been input or less, or extracts a candidate molecule showing data of binding amount during low activity of less than the threshold that has been input.

18. The screening device of claim 17, wherein
the extracting unit extracts a candidate molecule showing data of binding amount during activity of a threshold that has been input or more, or extracts a candidate molecule showing data of binding amount during activity exceeding the threshold.

19. The screening device of claims 16, wherein:
the immobilization membrane is formed on one side of a metal membrane and is not formed on the other side, and
obtaining the data of binding amount during activity and obtaining the data of binding amount during low activity are carried out by a process including using attenuated total reflection generated by irradiating a light beam to the opposite side of the metal membrane to that on which the immobilization membrane is formed.

20. The screening device of claim 19, wherein:
obtaining the data of binding amount during activity and obtaining the data of binding amount during low activity are carried out by a process including supplying the candidate molecule to a metal membrane on which is formed the immobilization membrane on which the target molecule is not immobilized, the data being based on reference data obtained from the amount of attenuated total reflection generated by irradiating the light beam to the opposite side of the metal membrane to that on which the immobilization membrane on which the target molecule is not immobilized is formed.
